SEQUENCE LISTING (3 sequences)

SEQ ID NO: 1 - DNA, 30 bp, Artificial Sequence
  ODN monomer with a central probe sequence
cacacagatactggcaagcttgagtttttt

SEQ ID NO: 2 - DNA, 12 bp, Artificial Sequence
  A coupler that can hybridize to opposite ends of two monomers.
tgtgtgaaaaaa

SEQ ID NO: 3 - DNA, 18 bp, Artificial Sequence
  Exemplified probe sequence.
gatactggcaagcttgag

(19) United States
(12) Patent Application Publication
(10) Pub. No.: US 2005/0112636 A1
(43) Pub. Date: May 26, 2005

(54) Polymeric nucleic acid hybridization probes

(75) Inventors: Richard A. Hurt, Oak Ridge, TN (US); Tom J. Whitaker, Oak Ridge, TN (US)

Correspondence Address: BIRCH STEWART KOLASCH & BIRCH, PO BOX 747, FALLS CHURCH, VA, 22040-0747, US

(73) Assignee: ATOM SCIENCES, Oak Ridge, TN (US)

(21) Appl. No.: 10/947,310

(22) Filed: Sep. 23, 2004

Related U.S. Application Data
(60) Provisional application No. 60/504,991, filed on Sep. 23, 2003.

(51) Int. Cl. C12Q 1/68; C07H 21/04

(52) U.S. Cl. 435/6; 536/24.3

(57) ABSTRACT

A novel polymeric nucleic acid probe improves detection sensitivity and specificity in a variety of hybridization platforms. The probe is made up of multiple short nucleic acid sequences (referred to as monomers) attached together to form a long polymeric probe for use in hybridization applications. For applications requiring immobilization of the probes to a surface, the polymeric probes are similar to long DNA probes in that they can be immobilized to a variety of surfaces without need for a chemical modification to the end of the probe. Because target nucleic acids hybridize to the relatively short monomers in the polymeric probe, the polymeric probes are more specific than long DNA probes. In addition, polymeric probes also improve the signal-to-background ratio by increasing the number of accessible monomer oligonucleotide probes immobilized per unit area on a surface.

### FIELD OF THE INVENTION

This invention is related to the field of nucleic acid hybridization. This includes hybridization of deoxyribonucleic acid (DNA) or ribonucleic acid (RNA) targets to probes having a known sequence for a wide range of applications, including: clinical diagnostics, clinical screening, genotyping, pathogen detection, pathogen identification, detection of specific genes, gene expression studies, medical applications, and detection of polymorphisms.

### BACKGROUND OF THE INVENTION

DNA and RNA

Genetic information is contained within the sequence of four bases (adenine [A], guanine [G], thymine [T], and cytosine [C]) in deoxyribonucleic acid (DNA). Similarly, there are four bases in ribonucleic acid (RNA), A, G, C and Uracil (U). In both DNA and RNA, these bases are attached to a sugar-phosphate backbone. This backbone has a structural directionality, with one terminus specified as the 5′ end and the other being the 3′ end. Unless otherwise specified, DNA sequences are, by convention, written from the 5′ end first. Thus, AGA-TCG-GTC is equivalent to 5′-AGA-TCG-GTC-3′. Furthermore, when two single strands of DNA bind (hybridize) to form a double-stranded DNA (hybrid), they do so in an antiparallel fashion, with the 5′ to 3′ direction in one strand being 180° from the 5′to 3 direction in the other strand. The most stable hybrids are formed when the sequence in one strand is complementary to the sequence in the other strand. A is complementary to T and G is complementary to C in DNA/DNA hybrids; A is complementary to U and G is complementary to C in DNA/RNA hybrids. This allows sequence information to be obtained about target nucleic acids by testing if stable hybrids form with probe nucleic acids for which the sequence is known. Several parameters, such as the length of the hybrid, degree of complementarity, position of any mismatches, G-C content, pH, and salt concentration all affect the stability of the resulting hybrid. In general, the stability of a short hybrid is more affected by a small number of mismatches than is the stability of a long hybrid.

Other Nucleic Acids

Peptide Nucleic Acid (PNA), [Egholm et al., U.S. Pat. No. 6,451,968] is a synthetic analog of DNA that has been used successfully as a replacement for DNA in hybridization and polymerase chain reaction technologies [see Ganesh et al., Current Org. Chem., 4 (9):931 (2000)]. PNA/DNA duplexes and PNA/RNA duplexes are generally more stable than are the corresponding DNA/DNA or DNA/RNA duplexes [Jensen et al., Biochemistry, 36:5072 (1997)]. A number of chemical backbone modifications of PNA have been prepared with varying success in their ability to mimic DNA in hybridization technologies [Ganesh et al., Current Org. Chem., 4(9): 931 (2000)]. The structure of the PNA backbone does not allow standard enzymatic ligation techniques but chemical methods have been developed. Another modification to native nucleic acids involves linking the 2′ oxygen and 4′ carbon in the sugar backbone. The product of this modification has been named “locked nucleic acid” or LNA. The furanose ring of LNA is locked in a C3′-endo conformation, and this leads to extremely stable LNA/DNA and LNA/RNA duplexes [Petersen and Wengel, Trends Biotechnol., 21: 74 (2003)].

Hybridization to Immobilized Probes

Hybridization experiments measure the degree of genetic similarity between nucleic acids of different origins. Often, these experiments are done with one nucleic acid of known sequence, which is referred to as the probe, and a nucleic acid that is the object of the investigation, which is referred to as the target. Hybridization experiments can be conducted in solution but this limits the number of simultaneous probe sequences that can be used. To overcome this limitation, probes with different sequences can be immobilized to different positions on a solid surface, thus enabling a high degree of multiplexing. These hybridization arrays (sometimes called DNA microarrays, genosensors, gene chips, etc.) are considered by many researchers to be the best method to determine if a specific sequence of DNA or RNA exists in a sample. The probes can be short oligodeoxynucleotides (ODNs), which are typically created by chemical synthesis, or longer sections of DNA, which are typically created by cloning or by duplicating DNA using the polymerase chain reaction (PCR) or other amplification techniques. Information about the sequence of the target nucleic acid is obtained by allowing single-stranded target nucleic acid to hybridize to the probes. When the two strands are perfect complements, the resulting hybrid is most stable. Even a single mismatch will significantly reduce the stability of a 25-bp hybrid [Wang et al., 1995] and thus, under the proper conditions, which are collectively referred to as the “stringency”, the existence of a stable hybrid at a particular probe site after hybridization indicates the existence of a complementary sequence in the target nucleic acid. Thus, under the appropriate stringency, the existence of a stable DNA/DNA hybrid at the site of a probe with sequence AGA-TCG-GTC would indicate that a section of the target has the sequence GAC-CGA-TCT. The existence of the stable hybrid is usually determined by attaching a label to the target DNA and detecting that label after the hybridization reaction. Practitioners skilled in the art will recognize that ribonucleic acid (RNA) targets can also be probed by this type of array without any modification to the array or to the probes. Similarly, it will be recognized that the probes may be made from DNA analogs, such as peptide nucleic acids [Egholm et al., U.S. Pat. No. 6,451,968], or chemically modified DNA, such as locked nucleic acids [Petersen and Wengel, Trends Biotechnol., 21: 74 (2003)].

Site-specific sequence immobilization in a hybridization array allows a large number of probe sequences to be employed on a single substrate to simultaneously test a target nucleic acid. The advantage of this can be seen in the example of pathogen detection. For pathogen detection and characterization, toxin-encoding gene sequences, sequences associated with toxin production and delivery, sequences related to virulence factors, and antimicrobial resistance genes could be targeted simultaneously to improve the certainty of a diagnosis. Diagnosis of viruses would rely on multiple probes that aim to identify sequence structures present in the virus genome. Therefore, if a large number of pathogens are to be simultaneously surveyed in a diagnostic procedure, an even larger number of hybridization reactions are required. Microarrays offer the ability to perform these reactions simultaneously. The parallel nature of DNA arrays also allows control sequences to be tested under identical conditions with the other probes. Control sequences are sequences that are complementary to sequences that are known to be in the target nucleic acid (positive control) or complementary to sequences that are known to be absent in the target nucleic acid (negative control).

Long Probes

Long probes are probes that are attached to the substrate surface through multiple attachments. A long probe can be attached, for example, to poly-L-lysine coated glass slides and cross-linked using ultraviolet radiation. Several other coatings, such as amine or epoxy coatings, can also be used. In coatings with primary amine groups (R—NH₂), for exmaple, the amines carry a positive charge at neutral pH, allowing attachment of long DNA through the formation of ionic bonds with the negatively charged phosphate backbone of DNA. Exposure to ultraviolet light or heat will induce covalent bonds that supplement the electrostatic interaction. As the DNA is bound at multiple locations along its length, specific sections of the DNA may not be available for hybridization to the target. However, the length of these DNAs makes the hybrids quite stable, even with mismatches and some unavailable sections on the probe. Because the long DNAs attach to the substrate along the length of their structure, end-modifications, which are required for immobilization of short DNAs, are not needed for long DNA probes. This represents a significant savings in cost and complexity over the short synthesized DNA probes. Another advantage is that sequence-dependent variations in stability, caused by the fact that G-C bonds are stronger than A-T bonds, tend to average out in long DNA. However, the instability caused by a single-base mismatch is also less significant in a long probe, making allele-specific hybridization difficult and preventing the use of long probes in applications directed at polymorphism characterization. Long probes are better suited for applications that monitor gene expression. Another problem with long probes is that extensive effort is required to prepare clones or PCR products, and sequences generated by PCR for expression monitoring are often limited to those that can be reliably amplified.

Short Probes

The primary advantage of short ODN probes is that a single-base mismatch destabilizes a short hybrid more than it does a long hybrid. This property can be exploited for applications that require allele-specific hybridization, such as determining single-nucleotide polymorphisms (SNPs). The fact that hybrid stability decreases with shorter lengths, along with the fact that longer probes assist in resolving internal structure in long targets, prevents the use of ODN probes shorter than about 8 bases.

Immobilization of short probes typically requires expensive end-modifications to the ODN. Design of chemistries for attachment of oligonucleotides to solid supports has become a major research focus in microarray development technology and there are a number of commercially available activated substrates that perform very well for ODN immobilization. A photolithography technique has been used to synthesize oligonucleotide probes in situ. This technique enables extremely high-density ODN probe arrays [Fodor et al., Science, 251: 767 (1991)]; however, microarrays prepared using this approach are very expensive. The photolithographic method is also less appropriate for intermediate- and low-density arrays or for situations where a large number of replicate experiments are needed. Chemistries utilizing 5′- or 3′-alkylamines react well with aldehyde, epoxide, and amine modified surfaces [Dolan et al., Nucleic Acids Res., 29: e107 (2001); Guo et al., Genome Res., 12: 447 (2002); Beattie et al., Mol. Biotechnol., 4: 213 (1995)]. Thiol derivatized ODNs have been shown to attach to mercaptosilanated or gold-coated surfaces (Herne and Tarlov, J. Am. Chem. Soc., 119: 8916 (1997); Rogers et al., Anal. Biochem,. 266: 23 (1999)].

Other chemistries that have also been shown to work well involve unstable intermediates such as N-hydroxy succinimide esters and/or complex chemical manipulations [Kwiatowski et al., Nucleic Acids Res., 27:, 4710 (1999); Shchepinov et al., Nucleic Acids Res., 27: 3035 (1999); Strother et al., Nucleic Acids Res., 28: 3535 (2000)].

The packing density of ODN probes on a solid support is an important consideration [Steel et al., Biophysical J., 79: 975 (2000); Peterson et al.,. Nucleic Acids Res., 29: 5163 (2001)]. Excessive packing density and proximity of the ODN probe to the surface reduces hybridization signal because of steric and crowding effects [Southern et al.,. Nature Genet., 21 supplement: 5 (1999)] and/or electrostatic repulsion due to the concentration of negative charges of the DNA backbone at DNA modified surfaces [Steel et al., (2000); Herne and Tarlov, (1997)]. The end modification often includes a molecular chain that serves as a spacer between the ODN and the attachment moiety [Schepinov et al., Nucleic Acids Res., 25: 1155 (1997)]. This allows the tethered ODN probe to extend farther from the surface and increases the hybridization signal. To further increase sensitivity, porous polyacrylamide matrices or “gel pads” have been developed [Livshits and Mirzabekov, Biophys. J., 71: 2795 (1996)]. It has been found that hybridization using the “gel pad” approach is more sensitive because of a higher probe concentration per unit area and improved probe accessibility [Drobyshev et al., Gene, 188: 45 (1997)]. Improved mismatch discrimination has been suggested to result from the approximation of solution-phase hybridization conditions for the gel pad approach [Drobyshev et al., (1997); Livshits and Mirzabekov, (1996)]. Immobilization on porous substrates has also been used [Cheek et al., Anal. Chem., 73: 5777 (2001)]. Although methods have been developed for the co-polymerization of polyacrylamide and activated ODN probes, the preparation of gel pad arrays is tedious and technically challenging. The low porosity of polyacrylamide affects hybridization kinetics and imposes constraints on the length of the targeted nucleic acid sequences [Proudnikov et al., Anal. Biochem., 259: 34 (1998)].

Polymeric Probes

A polymer is a molecule that is composed of multiple copies of a smaller molecule called the monomer. The monomers are covalently bonded together to form the polymer. For this application:
a monomer is a specific nucleic acid sequence of at least eight bases;
a polymer can be a homopolymer consisting of a multiple copies of a single monomeric sequence or it can be a copolymer that consists of multiple copies of two or more monomers with different sequences;
the order of the different monomers in a copolymer can vary;
monomers can be directly bound end-to-end or molecular linkers can be used to bind the monomers together;
molecular linkers can be identical throughout the polymer or may vary in composition and size.

Methods to Construct a Polymeric Probe—Ligation Methods

One method of forming polymeric probes is to ligate monomeric units using a DNA ligase. DNA ligase is an enzyme that repairs broken strands of DNA. There are a large number of ligases and ligation techniques. Most DNA ligases repair a nick in one strand of double-stranded DNA; these ligases typically require that the 5′ end of the nick is phosphorylated and that the adjacent 3′ end at the other side of the nick has a hydroxyl group available. Researchers have used this method to search for mutations by ligating sequences that are complementary to the wild-type sequence and then using gel electrophoresis to determine the length of the resulting strand. A mutation would cause one of the sequences to bind less effectively and cause a fraction of the ligation products to be shorter than the full tested length [Yager, et al., U.S. Pat. No. 6,025,139]. Another closely related method involves a recursive directional ligation to form a synthetic gene [Meyer and Chilkoti, Biomacromolecules, 3(2): 357 (2002)]. In this technique, a sequence of double-stranded DNA is repeatedly ligated to prepare protein-based polymers. These techniques could be modified to prepare the polymeric probes of this application. However, the complementary sequence would need to be removed before the polymeric probe could be used in hybridization technologies.

Methods to Construct a Polymeric Probe—Strand Displacement Amplification with a Circular Template

Rolling Circle Amplification (RCA) is an isothermal amplification technique for nucleic acids. RCA uses a circular DNA template and a specialized polymerase that displaces the primer and extension product as it travels around the template to generate a long, single-strand DNA product that is a tandem repeat of the circle's sequence complement. The process has been patented for the purpose of amplifying reporter molecules that have a “specific binding molecule” that selectively binds to the target (Lizard, P. M., U.S. Pat. Nos. 5,854,033; 6,210,884). By eliminating the “specific binding molecule” in this method, the technique would be an alternate method for forming the polymeric probes.

### SUMMARY OF THE INVENTION

The invention is a polymeric nucleic acid hybridization probe made up of multiple copies of a nucleic acid probe sequence, which is complementary to a sequence of interest in the target nucleic acid. The monomeric unit in the polymer may include one or more linker moieties attached at either or both ends of the probe sequence. Multiple copies of the monomeric units are bound together either directly or via additional linkers moieties that may vary within the polymer. This forms a long chain polymeric probe that has many of the conveniences of long DNA hybridization probes, such as not requiring end-modification for immobilization on hybridization array surfaces, and many of the attributes of short DNA hybridization probes, such as the ability to discriminate against single-base mismatches.

### BRIEF DESCRIPTION OF THE DRAWINGS

FIG. 1. This diagram shows an example of the ligation of two identical ODN monomers in one embodiment of the invention. The monomers are depicted on the top line separated by a vertical bar. The monomers have a central probe sequence, which is underlined, surrounded at each end by 6-mer linkers. The coupler that will hybridize to these linkers is depicted on the second line. This coupler is depicted with the 3′ end on the left in order to show its binding position to the monomers. A large separation is drawn between the A and G bases in the coupler so that the bases line up with their complement in the two monomers. This separation is strictly for depiction of the alignment and the actual separation between these two bases would be the same as between other adjacent bases in the coupler. The dissociation temperature, T_{d}, for this particular coupler duplex after ligation is calculated to be about 29.7° C., allowing removal of the coupler after ligation without denaturing the ligase.

FIG. 2a-2e. Exemplified method of ligation. In this figure, the parts of the monomer are indicated by different thickness of the line; the probe sequence is shown with a thick line, the 5′ linker with a medium thickness line, and the 3′ linker with a thin line. The letter P at the end of the 5′ linker indicates phosphorylation of the 5′ end. Ligated nicks are indicated by a small, filled circle. Reaction A is the hybridization of the coupler to the linkers at the ends of the ODN, reaction B is the ligation, and C is an incubation at elevated temperature to dissociate the duplex and remove the coupler. FIG. 2a shows the reaction for an unphosphorylated monomer with itself and FIG. 2b shows the reaction for a phosphorylated monomer with itself. FIGS. 2c, 2d, and 2e show the possible reactions between, respectively, two unphosphorylated monomers, one unphosphorylated monomer and one phosphorylated monomer, and two phosphorylated monomers.

### DETAILED DESCRIPTION OF THE INVENTION

The invention utilizes linked nucleic acid monomers to form polymeric hybridization probes. The monomers are made up of at least a nucleic acid probe sequence that is designed to be complementary to sequences of interest that may be present in the target nucleic acid. The monomer may also have linker on either or both ends, each linker comprising a nucleic acid sequence or other molecular moiety or a combination of both. The polymeric probe can be attached to hybridization surfaces in the same manner as long DNA probes, binding at several locations along the polymeric chain. In between these binding locations, monomeric units will be available for hybridization to the target DNA. Blocking molecules can be used to bind to a portion of the surface, thus preventing polymeric probes from attaching at those sites and increasing the fraction of monomeric units in the polymeric probe that are available for hybridization. The length of the polymeric probe will allow many of the monomeric units to be located well away from the surface, providing conditions similar to solution-phase hybridization. This three-dimensional effect will also allow a larger density of monomeric units per unit surface area, increasing the number of targets that can be hybridized to probes at each attachment site.

In a one embodiment, probe monomers are assembled into polymeric probes using T4 DNA ligase and a complementary coupler DNA sequence. T4 DNA Ligase (similar to a number of other ligases) covalently joins 5′-phosphorylated to 3′-hydroxylated DNA termini at blunt or compatible cohesive ends of double-stranded DNA fragments. For ligation of single-stranded DNA, a complementary coupler must be added so that the T4 DNA ligase will function. A universal coupler can be used if the monomer is synthesized with linker sequences on the ends.

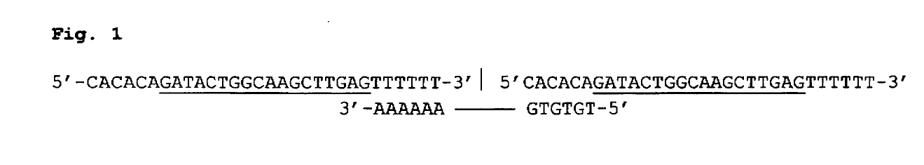

In this embodiment, the coupler sequence should be of limited length and have a low (G+C) content so that it can be easily removed following the ligation reaction. One step in the ligation reaction in this embodiment is shown for a specific probe sequence and specific linker sequences in FIG. 1. In this case, the exemplified probe sequence is GATACTGGCAAGCTTGAG. In the initial synthesis, a T₆ six-mer linker is attached to the 3′ terminus or the probe sequence and a CACACA six-mer linker is attached to the 5′ terminus of the probe sequence, forming a 40-mer that is used as the monomeric unit. Thus a TGTGTGAAAAAA coupler can hybridize to opposite ends of two monomers, connecting the two ends and forming a double stranded section with a gap (called a “nick”) between the two linkers. A standard ligase can be used to covalently bond the linkers across this nick.

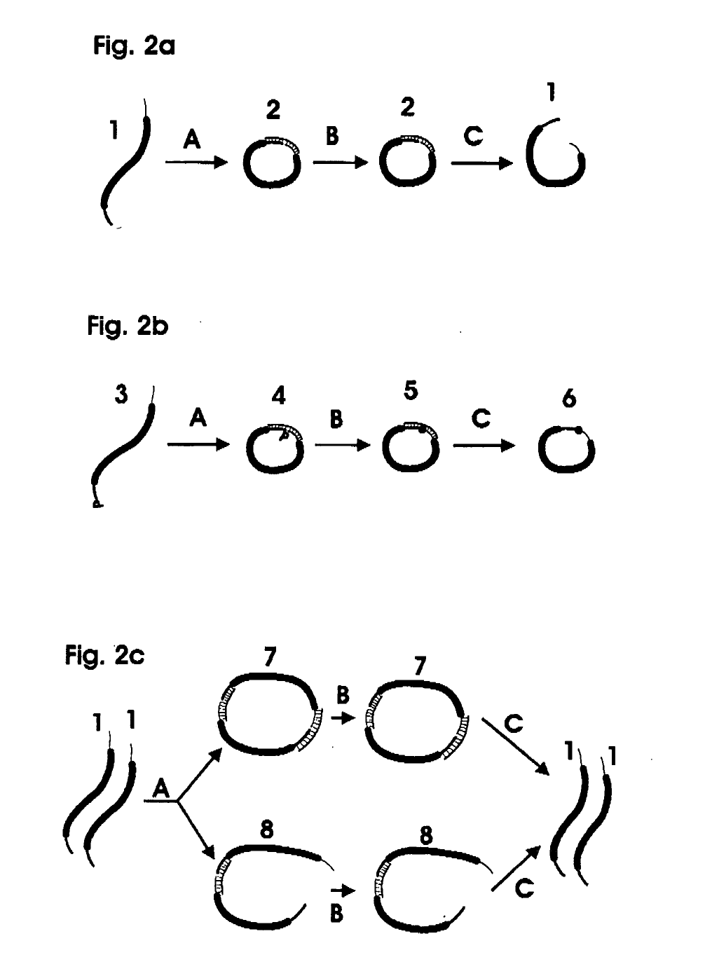

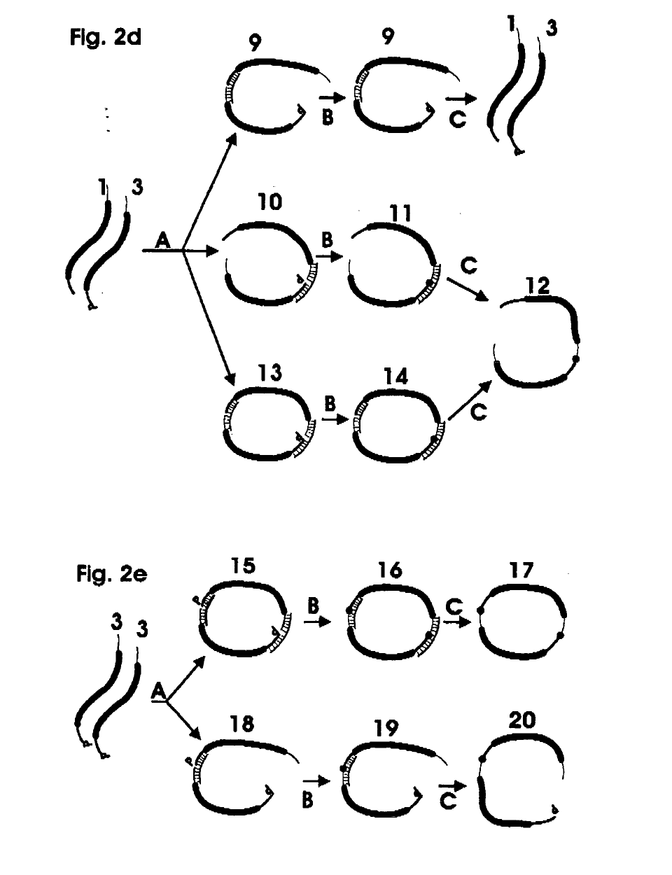

In standard syntheses, oligonucleotides typically are terminated with a hydroxyl group on the 3′-end but the 5′-end is generally not phosphorylated. Thus, phosphorylation is required before the ligase can be effective, and this can be accomplished using T4 Polynucleotide kinase or any of a number of means known to those skilled in the art. However, complete phosphorylation of the 5′-end is not desirable as shown in FIGS. 2a-2d. These figures show some of the possible ligation results with a mixture of phosphorylated and non-phosphorylated ODN monomers. One skilled in the art would recognize that the monomers depicted in these figures could also represent polymers that have previously undergone ligation. In FIGS. 2a-2d, the parts of the monomer are indicated by different thickness of the line; the probe sequence is shown with a thick line, the 5′ linker with a medium thickness line, and the 3′ linker with a thin line. The letter P at the end of the 5′ linker indicates phosphorylation of the 5′ end. Reactions and dissociation are indicated by arrows labeled with the letters A, B, and C. Reaction A is the hybridization of the coupler to the linkers at the ends of the ODN, reaction B is the ligation, and C is an incubation at elevated temperature to dissociate the duplex and remove the coupler. Except in the case of very short monomers, it is possible for a single monomer to have both ends hybridize to the same coupler molecule as shown in FIGS. 2a and 2b. FIG. 2a shows this reaction for an unphosphorylated monomer, 1. The coupling reaction results in a circular molecule, 2, that cannot be ligated by T4 DNA ligase. Therefore, when the coupler is removed in reaction C, the initial monomer is returned to its original, linear state, which can undergo further reactions. However, when a phosphorylated monomer, 3, undergoes the same coupling reaction (FIG. 2b), it gives 4, which can be ligated in reaction B by T4 DNA ligase to form a continuous, circular ODN with the coupler still attached, 5. After removal of the coupler in reaction C, the result is a circular, single-stranded ODN, 6. The circular molecule, 6, cannot participate in further reactions to form the desired long polymeric probes; however, it serves a useful purpose in applications requiring immobilization of the long polymeric probe to a surface because molecule 6 will attach to the surface and limit the number of surface locations to which the polymeric probes can bind. This increases the number of monomeric units in the polymeric probe that are available for hybridization to the target. The relative yield between these self-ligating reactions and ligation of two different molecules can be adjusted by increasing the concentration of the monomeric ODNs. It is noted that the circular molecule, 6, can be used as a template in rolling circle synthesis of very long polymeric probes, provided only that the starting molecule, 6, has the same sequence as the target DNA instead being complementary to the target DNA.

FIG. 2c shows the possible reactions between two unphosphorylated monomers, 1. The coupling reaction A can produce a fully circularized molecule, 7, or a linear molecule, 8. Neither 7 nor 8 can be ligated in reaction B because of the lack of a phosphate group, and in both cases reaction C returns the original linear monomers.

FIG. 2d shows the possible reactions between one unphosphorylated monomer, 1, and one phosphorylated monomer, 3. The coupling reaction can produce either a linear molecule with a phophorylated 5′ terminus, 9, a linear molecule with an unphosphorylated 5′ terminus, 10, or a circular ODN, 13. Molecule 9 cannot be ligated in reaction B and therefore reaction C returns the two starting monomers. Molecule 10 can be ligated to form a linear molecule, 11. After removing the coupler in C, the result is a linear, single-stranded polymeric probe, 12 made up of two monomers (N=2). In the case where the coupler reaction A forms a completely circularized molecule, 13, only one of the coupled sections has a phosphate and the ligation reaction joins only that phosphorylated section to form a circular molecule, 14. Reaction C removes the coupler and results in the same linear polymeric probe, 12, as the previous example. Molecule 12 can undergo further reactions to make longer polymeric probes. So in all cases with the reaction of one phosphorylated ODN with one unphosphorylated ODN, the result is either the starting material or a polymeric probe that can undergo further reactions.

FIG. 2e shows the possible reactions between two phosphorylated monomers, 3. The coupler reaction A results in two possible products, a fully circularized molecule, 15, and a linear molecule, 18. As all the 5′ ends are phosphorylated, ligation reaction B leads to respectively, a circular ODN, 16 and a linear ODN, 19. After removing the coupler in C, the products are respectively a single-stranded, circularized ODN, 17, and a single-stranded linear ODN, 20. Molecule 17 is similar to molecule 6 in that it is not available for further reactions but may be useful in blocking sites from the polymeric probes. Molecule 20 has a phosphorylated 5′ end and, if combined with a phosphorylated monomer or polymer in another reaction, could result in a terminal circularized product. However, if molecule 20 combines with an unphosphorylated monomer or polymer, it forms a larger polymer that will not circularize.

Reactions A and B can be run simultaneously at the same temperature. The decoupling reaction, C, requires a higher temperature and therefore a thermal cycling procedure can be used in the preferred embodiment to repeat the reactions and increase the length of the polymeric probes. The length of the coupler molecule and the G-C content can be designed so that the hybrid it forms with the probe molecules dissociates below the denaturing temperature of T4 DNA Ligase.

Even with the limitations imposed by circularization, the polymeric probes work significantly better than the standard monomeric probe. Experiments demonstrated that the hybridization signal from 1-femtomolar dye-labeled E. Coli target DNA hybridized to immobilized complementary probes at 50° C. improved by a factor of at least three when polymeric probes were used as compared to monomeric probes. This was true for all of the three concentrations of polymeric probe tested (12.5, 6.25, and 3.125 micromolar); monomeric probes were at their optimum concentration (50 micromolar). Note that the polymeric probe concentration is given in terms of the monomeric unit so that 12.5 micromolar polymeric probe has 4 times less monomers than an equal volume of 50 micromolar monomeric probe.

Other Ligation Reactions—In a second embodiment, a thermostable DNA ligase such as Ampligase, Thermophage™ single-stranded DNA ligase, or Tfi ligase is used instead of T4 DNA ligase so that higher temperatures can be used in process C without denaturing the ligase. In a third embodiment, direct ligation of monomeric probe units can be accomplished using T4 RNA ligase [Tessier et al., Anal. Biochem., 158: 171 (1986)]. This reaction directly links the ODN probes without the use of a coupler. Even though linkers are not required at the ends of the probe sequence as they are in the case of T4 DNA ligase, a linker on at least one terminus serves to separate the probe sequences in the polymeric probe and reduce steric hindrance to hybridization caused by a hybrid formed at an adjacent monomer site. In yet another embodiment, non-enzymatic (chemical) methods [Xu and Kool, Nucleic Acids Res., 27: 875 (1999); Liu and Taylor, Nucleic Acids Res., 26: 3300 (1998)] are used to ligate the monomeric probes. Again, linkers on at least one end of the probe sequence could be used to reduce steric hindrance to the hybridization reaction.

Monomers with Different Sequences—In another embodiment, monomers with different sequences could be ligated so that the polymeric probe becomes a copolymer, which incorporates multiple sequences. This could be useful if the user desires to know whether any of several possible SNPs are in the target DNA.

Preventing the Coupler Sequence from Being Ligated—In another embodiment, the 3′ end of the coupler sequence can be protected by using a dideoxynucleoside triphosphate to add the final base to the 3′ terminus of the sequence. This can be accomplished by any of a number of nucleotide extension reactions known to those skilled in the art. The 5′ end of the coupler sequence is already protected from ligation to any other nucleic acids because it is not phosphorylated.

Circularization—In another embodiment, after the desired length of the polymeric probe has been achieved, T4 Polynucleotide Kinase can be used to phosphorylate the 5′ end of the polymeric probe. This, followed by the coupler hybridization and ligase, will cause a large number of the polymeric probes to circularize. Although circularization (ligation of opposite ends of the same polymeric probe molecule) should be avoided in the early thermal cycles because it prevents further increase in the length of the polymeric probe, it may be desirable after the polymeric probe has reached an acceptable length.

Rolling Circle Amplification—In another embodiment, rolling circle amplification (RCA) can be used to create a very long polymeric probe with the only limitation being that the entire molecule must be DNA. RCA uses a strand-displacement polymerase, such as φ29 polymerase, with a circularized oligonucleotide template. The product is a very high molecular weight, single-stranded oligonucleotide composed of multiple tandem repeats of the circle's complement. Circularized template for the RCA reaction is made in a ligation reaction similar to that described above except the initial monomeric sequence would be identical to the target DNA sequence so that each monomeric component of the RCA product would be complementary to the target sequence. The circularized template can consist of circles with different numbers of monomeric units but all can be primed by the same molecule and all give the same product. Following the RCA reaction, the product can be sheared to the desired size.

Exemplified Embodiments

Synthesis of Polymeric Probes using Ligation—T4 DNA ligase along with a coupler molecule with the sequence (TG)₆ was used to ligate E. coli monomeric probe sequences that were flanked by (CA)₃ at both the 5′ and 3′ termini in a 16-h room temperature ligation reaction. A β-proteobacterial consensus sequence with similar terminal additions was likewise ligated. The products were electrophoresed in a 6% denaturing polyacrylamide gel at 250 V×2 h using a BioRad Protean™ II xi electrophoresis cell. In both cases, the resulting electrophoretic profiles of the polymeric probes showed a pattern of two bands occurring at each increment (N=2 to N=10), consistent with bands of linear and circularized DNA taken by others (e.g., Prokaria's website at prokaria.com).

Ligated Polymeric Probe hybridization results—The ligated polymeric probes were serially diluted and printed onto a Superaldehyde™ slide. The 5′C₆-alkylamine modified consensus sequence monomers and similarly modified E. coli sequence monomers were printed using a 50-μM probe concentration in Micro Spotting Plus solution (Telechem), which was previously found to provide optimal results for depositing monomers. Three different concentrations of unmodified polymeric probes (12.5-, 6.25-, and 3.13-μM) were deposited in spotting solution [3×SSC, 1.5 M betaine]. It is important to note that concentrations for the polyprobes are given in terms of the monomeric unit; so equal volumes of equal concentrations of the monomeric probe and polyprobe have an equal number of monomeric units. Optimal immobilization conditions for polymeric probes may varied as necessary according to polyprobe length. The printed monomeric probes and polyprobes were allowed to react with the surface overnight. Hybridization was performed using 100 mM sodium phosphate, 1 × Denhardt's reagent, 0.3% SDS, and 1 pmol of Cy5-labeled E. coli probe complement, and 1 pmol of Cy3-labeled consensus probe complement at 45° C. for 12 h. These hybridization conditions were selected for optimum selectivity and signal intensity for probes deposited as monomers. After hybridization, the slide was washed at room temperature and fluorescence images were obtained with a Perkin-Elmer ScanArray imager. These results showed that polymeric probes perform at least as well as the C₆-alkylamine modified monomeric probes under these conditions, even though the monomer concentration in the polymeric probe is significantly lower than that of the C₆-alkylamine modified monomeric probes. When the hybridization reaction described in the previous paragraph was repeated except with higher stringency, (1 fmol target, 50° C. hybridization temperature), the average fluorescence intensity for the polymeric probes was significantly stronger than the monomers for the E. coli sequence.

Synthesis of Polymeric probes using Strand Displacement Amplification—We have synthesized long polyprobes using strand displacement amplification with ligated polymeric probes as a template. The reactions used φ29 DNA polymerase (New England BioLabs), short ligated polymeric probes from a reaction identical to that described previously as a template, and the (TG)₆ coupler molecule as primer. The reaction conditions were as described by [Dean, et al. Comprehensive human genome amplification using multiple displacement amplification. Proc. Natl. Acad. Sci. USA 99:5261-5266 (2002)]. The Strand Displacement Amplification (SDA) reaction produced long, single-stranded polymeric probes. To show this, we compared gel electrophoresis profiles of the sheared SDA reaction product with other DNAs. Shearing was necessary because the unsheared product was too large to enter the gel. The sheared SDA reaction product's electrophoresis profile matched that of sheared single-stranded DNA but not that of sheared double-stranded DNA. (all were sonicated with a Misonix™ Cell Disruptor for 60 s at power level 2).

Shearing SDA reaction products for only 5 s produced a size distribution from about 500 bp to 8 Kbp, corresponding to about N=20 to 270. The sheared products were immobilized onto microarrays and hybridization reactions were performed with E. coli and consensus sequence labeled targets. Fluorescence imaging gave signals at the appropriate location, proving that the SDA products have the correct sequence for polymeric probes.

1. A single-stranded polymeric probe comprising multiple copies of one or more sequences of single-stranded nucleic acid (monomers), which are joined together, wherein;
the monomers are at least 6 nucleotides in length;
the monomers are joined either directly end-to-end or to opposite ends of a molecular linker that may or may not include other monomers with a different sequence;
said polymeric probe contains at least 4 copies of said monomer; and
said polymeric probe has at least one probe sequence that is known to be complementary either to a potential sequence in a target nucleic acid, to a positive control sequence in the nucleic acid, or to a negative control sequence that is absent in the target nucleic acid.

2. The polymeric probe of claim 1, wherein the monomeric nucleic acid is selected from the group consisting of synthetic oligodeoxynucleotides (ODNs), peptide nucleic acid (PNAs), locked nucleic acids (LNAs), and sections of isolated DNA formed using DNA amplification techniques.

3. The polymeric probe of claim 1 wherein said monomeric sequences of single-stranded nucleic acid include an additional sequence of nucleic acid on one or both ends of said probe sequence.

4. The polymeric probe of claim 1 wherein said monomeric sequences of single-stranded nucleic acid are linked on one or both ends of said probe sequence by a molecular linker that is not a nucleic acid.

5. The polymeric probe of claim 1 wherein the polymeric probe is a homopolymer.

6. The polymeric probe of claim 1 wherein the polymeric probe is a copolymer having two or more different sequences for the monomeric units.

7. The polymeric probe of claim 1 wherein said polymeric probe is linear.

8. The polymeric probe of claim 1 wherein said probe polymeric probe is circular.

9. A method of forming the probe of claim 1 which comprises repeatedly joining a phosphorylated 5′ terminus of a monomer or polymer to a 3′ end of a polymer or monomer sequence that is not phosphorylated on the 5′ end.

10. The process of claim 9 wherein joining is accomplished by hybridizing a complementary coupler nucleic acid to a sequence on the 3′ of one monomer or polymer and to a sequence on the 5′ end of another monomer or polymer, so as to form a double-stranded section with a gap (“nick”), and ligating the ends of the nucleic acids that form the gap.

11. The method of claim 10, wherein a ligase enzyme is used to ligate the ends of the nucleic acid.

12. The method of claim 11, wherein the ligase enzyme is an enzyme that requires a phosphate on the 5′ terminus and a hydroxyl on the 3′ terminus of the nucleic acid.

13. The method of claim 12, wherein the ligase enzyme is T4 DNA ligase, T7 DNA ligase, Tfi DNA ligase, Ampligase, Tsc DNA ligase, or Chlorella virus PBCV-1 DNA ligase.

14. The method of claim 10, wherein a universal coupler is used for different probe sequences by terminating the 3′ ends and 5′ ends of the probe sequences with a nucleic acid linker having at least 4 bases in length; wherein the nucleic acid linker for the 5′ end is the same or different in length, sequence, or both from nucleic acid linker for the 3′ end.

15. The method of claim 10, wherein the ligation is repeated by using a thermal cycle, having a reaction mixture temperature that cycles from a value that permits the coupler to hybridize, to a value that permits ligation, to a value that dissociates the hybridized coupler molecule from the ligated polymer.

16. The method of claim 15, wherein said thermal cycle is repeated multiple times to increase the length of the polymer.

17. The method of claim 15, wherein ligase denaturing is avoided by using a coupler that has a length and G+C content that to will form a hybrid that will dissociate at a temperature where at least 50% of the ligase remains effective after 30 minutes of incubation.

18. The method of claim 15, wherein ligase denaturing is avoided by using a thermally stable ligase.

19. The method of claim 9, wherein the monomers and/or polymers are joined using a ligase that can directly link the single-stranded monomers and polymers together without the need of a coupler molecule.

20. The method of claim 19, wherein the ligase enzyme is T4 RNA ligase or Thermophage™ single-stranded DNA ligase.

21. The method of claim 9, wherein the monomers and/or polymers are joined using a non-ligase enzyme method.

22. A method of making a nucleic acid array, which comprises immobilizing the polymeric probes of claim 1 onto a solid substrate surface at different discrete locations, wherein each polymeric nucleic acid probe is made up of one or more monomer sequences.

23. The method of claim 22, wherein the solid substrate is glass, silicon, nylon, polyacrylamide gel, Teflon™, or metal.

24. The method of claim 22, wherein the solid substrate surface is coated with a moiety to enhance nucleic acid immobilization.

25. The method of claim 22, wherein the solid substrate surface is coated with an epoxide, aldehyde, poly-L-lysine, nylon, amino, carboxylate, or other moiety that enhances binding chemistry.

26. The method of claim 22 wherein the probes are covalently cross-linked to the solid substrate surface using ultraviolet light, heat, or reactive chemistry.

27. A nucleic acid array comprising polymeric probes of claim 1 immobilized onto a solid substrate surface at different discrete locations, wherein each polymeric nucleic acid probe is made up of one or more monomer sequences.

28. A method of assaying for a target nucleic acid which comprises, contacting a sample that contains the target nucleic acid with a nucleic acid array of claim 27, hybridizing the immobilized polymeric probes with the target nucleic acid; and detecting hybridization between the polymeric probes and target nucleic acid.

29. The method of claim 28, wherein the detection is done using radiological labeling, fluorescent labeling, bioluminescent labeling, chemiluminescent labeling, electrical detection, or mass spectrometry of labeled or unlabeled target nucleic acid.

30. The method of claim 28, wherein hybridization information obtained from the assay is used to determine if a specific nucleic acid sequence exists in the sample.

31. The method of claim 28, wherein hybridization information obtained from the assay is used to detect nucleic acid polymorphisms.

32. The method of claim 28, wherein the hybridization information from the assay is used to detect and/or identify a pathogen.

33. The method of claim 32, wherein the pathogen is from a bacteria, virus or fungus.

34. The method of claim 28, wherein the hybridization information is used to diagnose a disease.

35. The method of claim 28, wherein the hybridization information is used to monitor the efficacy of a disease treatment.

36. A method of forming a polymeric probe of claim 1, wherein the polymeric probe is prepared by direct oligonucleotide synthesis.

37. A method of forming a polymeric probe of claim 1, wherein the polymeric probe is prepared by strand displacement amplification of a circularized template.

38. The polymeric probe of claim 2, wherein the sections of isolated DNA formed using DNA amplification techniques is formed using polymerase chain reaction (PCR).
